(19) United States
(12) Patent Application Publication
(10) Pub. No.: US 2008/0214637 A1
(43) Pub. Date: Sep. 4, 2008

(54) Process for the Synthesis of Tetrazoles

(75) Inventors: Ljubomir Antoncic, Ljubljana (SI); Johannes Ludescher, Breitenbach (AT)

Correspondence Address: SANDOZ INC, 506 CARNEFIE CENTER, PRINCETON, NJ, 08540, US

(73) Assignee: LEK Pharmaceuticals D.D., ljubljana (SI)

(21) Appl. No.: 11/667,222

(22) PCT Filed: Nov. 11, 2005
(86) PCT No.: PCT/EP2005/011981

§ 371 Date: Nov. 19, 2007

(30) Foreign Application Priority Data
Nov. 11, 2004 (SI) P-0400307

(51) Int. Cl. A61K 31/41; C07D 403/02; A61P 9/12

(52) U.S. Cl. 514/381; 548/250; 548/253

(57) ABSTRACT

A process for the synthesis of tetrazol derivative has been developed which starts from a tetrazole derivative where acidic hydrogen atom has been replaced by a protecting group and the deprotection is performed with a catalytic amount of organic acid and can proceed in an aqueous solvent.

### FIELD OF THE INVENTION

The invention relates to a process for the synthesis of sartans that are the tetrazole derivatives of formula (I), where n is an integer form 0 to 2, preferably 1, and R is a suitable organic substituent, preferably containing nitrogen, more preferably optionally substituted imidazole, dihydroimidazole or benzimidazole and amine.

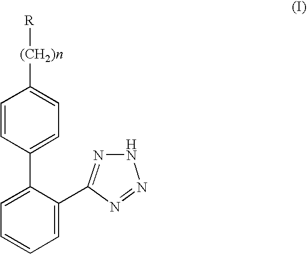

Among those tetrazole derivatives losartan, irbesartan, candesartan cileksetil, valsartan, olmesartan medoxomil (or salts thereof) are the active ingredient of modern antihypertensive drugs, the angiotensin II receptor antagonists.

### BACKGROUND OF THE INVENTION

Various substituted tetrazoles and the processes to prepare them have been disclosed for example in EP 253310, WO 9310106, EP 4543111 and EP 459136. A representative process to prepare losartan from trityl precursor has been disclosed in EP 1274702.

Known processes are generally performed in non-aqueous conditions for example with methanolic solution of gasous hydrochloric acid or generally with aqueous acid solution such as sulfuric or hydrochloric acid, or an excess of an organic acid.

Present invention discloses a new universal process for synthesis of aforesaid tetrazole derivatives allowing aqueous conditions and use of catalytic amounts of organic acids.

### DISCLOSURE OF THE INVENTION

The invention is embodied in a process for the synthesis of a compound of formula (I), where the R represents an optionally substituted imidazole, dihydroimidazole or benzimidazole or amine from a compound of formula (II) where Y is a protecting group.

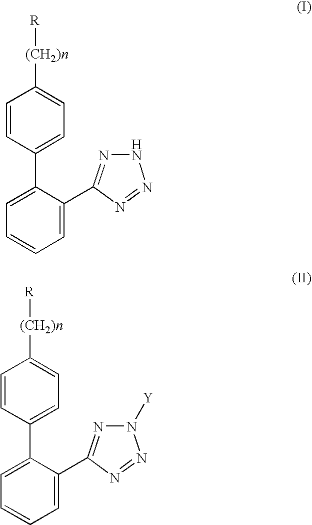

characterized in that the compound of formula (II) is reacted with preferably a catalytic amount of an acid, preferably an organic acid.

A specific aspect of the invention is a process for the synthesis of candesartan cilexetil characterized by comprising following steps:
a) preparing a solution of trityl candesartan cilexetil in an alcohol, or alcohol water mixture;
b) mixing said solution with an acid, until the substantially all trityl candesartan cilexetil is converted to candesartan cilexetil;
c) adding an amine to said solution of candesartan cilexetil;
d) (optionally) adding a water immiscible solvent;
e) (optionally) separating layers; and
d) isolating the candesartan cilexetil by addition of an acid.

Specifically in the synthesis of candesartan cilexetil the amine is ammonia or trialkylamine, preferably Et₃N; reaction is performed in an aqueous solution and acid used may be a mineral acid, preferably sulfuric or hydrochloric acid.

Another specific aspect of the invention is a process for the synthesis of a compound of formula (I), where the R is such that the compound of formula (I) is selected form a group consisting of losartan, irbesartan and olmesartan medoxomil or salts thereof starting from a compound of formula (IIb).

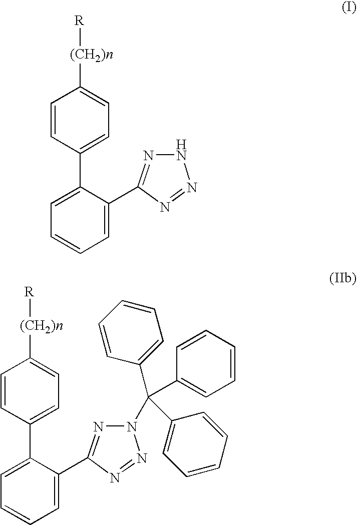

characterized in that the compound of formula (IIb) is dissolved in a solvent selected from chlorinated solvents, ethers; or alcohols; preferably methanol or ethanol, or mixture of them, optionally water is added and a catalytic quantity of a organic acids is used. Preferably a catalytic amount of from 1% to 75%, preferably to 50% molar ratio of organic acid to the starting compound may be used.

Further aspect of the invention is thus also the use of organic acid in a catalytic amount of from 1% to 50% molar ratio of organic acid to the starting compound in the process of deprotection of tetrazole derivative, specifically where tetrazole derivative is selected from losartan, irbesartan and olmesartan medoxomil or valsartan and preferably candesartan cilexetil.

Specifically the organic acid is selected from the group consisting of methane sulphonic acid, p-toluen sulphonic acid, pivalic acid, camphorsulphonic acid, trifluoracetic acid.

Additional aspects of the invention are the pharmaceutical composition comprising a compound produced as described. Specifically the compounds are losartan, irbesartan and olmesartan medoxomil or their salts, and candesartan cilexetil.

### DESCRIPTION OF THE INVENTION

The object of the present invention is an unified and robust process for deprotection of various substituted tetrazoles (removal of a protecting group on tetrazole), such as and preferably a removal of triphenylmethyl protecting group from tetrazole moiety of sartans in preparation of an active compound such as losartan, candesartan, irbesartan, valsartan and olmesartan and their esters such as medoxomil or cilexetil. Although use of organic and inorganic acids is contemplated, the essential element of the one particular embodiment of the process is the use of a catalytic quantity of an organic acids. Reaction takes place in the presence of water.

In accordance with the present invention, there is provided a pharmaceutical composition comprising tetrazole derivative prepared in accordance with our invention alone or in combination with another active ingredient such as hydrochlorotiazide and a pharmaceutically acceptable carrier comprising inactive ingredients such as fillers (diluents), binders, disintegrants, glidants, lubricants and other excipients.

Pharmaceutical composition in accordance with this invention can be embodied for example in form of tablet, capsules, pellets, granules and supozitories or their combined forms. Solid pharmaceutical compositions can be shielded, for example coated with the aim of increasing peletibility or regulating the disintegration or absorption.

### DETAILED DESCRIPTION OF THE INVENTION

Starting from the compound which in it's structure includes a tetrazole moiety where acidic hydrogen atom is substituted by a suitable protecting group (giving a derivative that is stable under the further reaction conditions), preferably by a triphenylmethyl (from thereon trityl), one can manufacture the tetrazoles which exhibit advantageous antihypertensive properties, and one can further prepare a salt of such tetrazole.

The starting compound is dissolved in a suitable organic solvent such as chlorinated solvent or an alcohol or an ether, for example in dichloromethan, chloroform, tetrahydrofuran, ethanol or methanol; preferably methanol. The concentration can for example lay in the range of 0.05g/ml to 0.5g/ml To the obtained solution water can be added.

In a specific embodiment, the solution will be an aqueous solution, preferably meaning containing per each mole of tetrazole one mole of water, more preferably 1.5 moles, still more preferably 2 moles.

The preferred starting compound of our invention is presented with formula (IIb), where n is an integer form 0 to 2, preferably 1, and R is a suitable organic substituent, preferably R is an optionally substituted amine, amide or at least one nitrogen containing heterocyclic system, such as optionally substituted benzimidazole or optionally substituted imidazole.

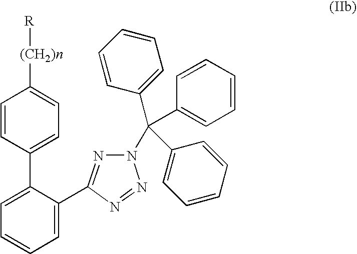

R can be more preferably selected from substituted valine, substituted cyclic saturated or unsaturated amine such as 1,3-diazaspiro [4.4]non-1-en-4-one or substituted benzimidazole or imidazole, such as C₁-C₄ alkyl and/or hydroxyl alkyl and/or halo and or substituted hetercyclo substituted imidazole and their oxidized or reduced derivatives, such as 2-ethoxy-1H-benzimidazole carboxylic acid or its ester or 2-butyl-4-halo-5-methanol-imidazole. In the most preferred embodiment the starting compound with formula (II) is a trityl protected sartan, such as irbesartan, candesartan, or candesartan cilexetil, losartan, valsartan or olmesartan, most preferably losartan and irbesartan, preferred compound is also olmesartan medoxomil.

To the obtained solution a catalytic quantity of an organic acid such as methane sulphonic acid, p-toluen sulphonic acid, pivalic acid, camphorsulphonic acid, trifluoracetic acid, ethanesulfonic acid, and benzensulfonic acid is added. Preferred acids are in one embodiment methane sulphonic acid and p-toluen sulphonic acid and if lesser reactivity is desired ethanesulfonic acid, and benzensulfonic acid. The reaction will proceed it the amount of acid will be higher than the amount of substrate, however it surprising that in the gram scale experiments a drop of acid is sufficient. The amount of acid will depend by the nature of the protecting group the reactions condition and particularly on the solvent, i.e. whether aqueous solvents are used. The molar amount of acid normally needed will be lower than molar amount of tetrazole preferably the amount of acid will be a catalytic amount which can be only few molar percent relative to the amount of tetrazole, most preferably above 1 or 4.5% and below 99%, preferably below 75%, and still preferably bellow 50% percent relative to the amount of tetrazole most preferably between 4.5 and 15%.

Reaction mixture is stirred for suitable period, which can be from few minutes to few days, preferably from 1 to few hours. The stirring time will depend on the reactivity of tetrazole and/or added acid and can be for trityl candesartan cilexetil or trityl olmesartan medoxomil about one hour, while for other tetrazoles of formula IIb up to 1 or more days. The stirring can be done at room temperature or at higher temperatures up to the temperature of reflux.

In one embodiment of our invention water or water and a chlorinated solvent such as dichloromethane are added to the above reaction mixture and pH may be adjusted by suitable alkali such as sodium hydroxide or sodium hydrogen carbonate.

The tetrazole may then be isolated by conventional means. The reaction mixture may be partially concentrated, for example part of the solvents removed, extracted with a suitable solvent, such as organic solvent such as diethylether, toluene or acetone, precipitated or crystallized with a suitable solvent and washed with suitable solvent such as ethyl acetate, acetone, ethanol, propanol. The obtained product can be further crystallized to produce suitable crystalline form or morphological variant.

Alternatively after the deprotection an amine, preferably ammonia or trialkylamine may be added to the above aqueous solution to afford an ammonium salt. Thereafter a solvent not miscible with water may be added and after separation of the layers the tetrazole is crystallized form the aqueous solution, preferably by addition of an acid.

Comparatively to the known process for deprotection of trityl losartan or trityl irbesartan using agueous HCl, the final work up is less complex and the product is substantially more pure, thus an additional purification step may not be needed.

In the process in accordance with our invention, one does not have to take care of anhydrous conditions, the amount of added acid will be minimal.

After the final work up the solid dosage forms comprising tetrazole derivative produced according to our process can be prepared by conventional method. Tablet can be for example manufactured by direct compression though wet granulation is another commonly used technique. In wet granulation at least one of the ingredients can be mixed or contacted with liquid and further processed to provide aggregates, the liquid can be partially or completely removed and optionally other or more of the same ingredients may be further added and solid dosage forms manufactured.

Tableting compositions may have in addition to active pharmaceutical ingredient few or many components depending upon the tableting method used, the release rate desired and other factors. For example, compositions of the present invention may contain inactive ingredients (excipients) which function as such as different fillers, binders, disintegrants, glidants, lubricants and excipients that enhance the absorption of drugs from gastrointestinal tract.

In one embodiment of the invention one can prepare film coated tablets by direct compression. Amorphous tetrazole derivative is mixed with lactose, microcrystalline cellulose, starch and mixture is sieved. A suitable glidant and/or lubricant is added and mixed again. Cores are tableted and coated with suitable suspension, for example comprising cellulose derivatives and titan dioxide in water or alcohol and the film coated tablets are polished with talc.

### EXPERIMENTAL PART

The identity of synthesized compounds have been confirmed by analytical methods such as HPLC, NMR, IR.

Following examples further illustrate the invention, They are provided for illustrative purposes only and are not intended to limit in any way the invention.

### Experiment 1 (Candesartan Cilexetil))

3 g (0.0035 mol) of trityl candersartan cilexetil was dissolved in a mixture of 9 ml of dichloromethane and 9 ml of methanol. Then 0.1 ml of water and 0.02 ml (cca one drop) of methanesulphonic acid (MSK) was added. Reaction mixture was stirred at room temperature for one hour 30 minutes, then a mixture of 6.3 ml of dichloromethane and 12.12 ml of water was added. pH was adjusted to 6.3 with a saturated solution of sodium hydrogen carbonate, organic layer was concentrated to the rest of 6.4 g. Then 6.3 ml of acetone was added and evaporated to drieness. 1 ml of ethanol was added to precipitate crystals. To the resulting mixture 22 ml of n-hexane were added and stirring was continued at room temperature for one hour. The separated crystals were filtered and washed with 10 ml of a mixture of ethanol: n-hexane=1:9. Product was dried at 60° C. to obtain raw product (1.98 g) which was crystallised from 19.8 ml of i-propanole to obtain pure candesartan cilexetil in Form 1. Yield: 1.64 g.

### Experiment 2 (Irbesartan)

23.25 g of trityl irbesartan was dissolved in 180 ml of methanol, 1 ml of water and 0.6g of p-toluensulphonic acid was added. Reaction mixture was stirred at the temperature of reflux for 4 hours and evaporated to dryness. Water was added, and ph was adjusted to pH12 with NaOH 30%. Reaction mixture was extracted with 150 ml of diethylether, 150 ml of toluene and 150 ml of diethyl ether successively. Layers were separated. pH of water layer was adjusted to pH 2 with 1 N HCl. Suspension was stirred at room temperature and filtered. Product was washed with 30 ml of ethyl acetate and vacuum dried at 50° C. Yield: 14 g

### Experiment 3 (Losartan)

4.7 g of trityl losartan) was dissolved in a mixture of 1.8 ml of THF (dichloromethane) and 18 ml of methanol. 0.2 ml of water and 0.06 g of p-toluenesulphonic acid (p-TSA) was added. Reaction mixture was stirred at room temperature for 6 days. A mixture of 12.6 ml dichloromethane and 24.24 ml of water was added, and then ph of reaction mixture was adjusted to pH 12 with NaOH 30%. Layers were separated, water layer was washed three times with 12.6 ml of dichloromethane and 15 ml of ethyl acetate was added. pH was adjusted to the value 3.6-3.8 with 1 N HCl and stirring was continued for another two hours at the temperature 5° C.-10° C. Product was filtered and washed with 10 ml of ethyl acetate, filtered again and dried at 60° C. Yield: 2.26 g

### Experiment 4 (Candesartan Cilexetil)

3 g (0.0035 mol) of trityl candesartan cilexetil was dissolved in a mixture of 9 ml of dichloromethane and 9 ml of methanol then 0.1 ml of water and 0.036 ml of trifluoroacetic acid was added. Reaction mixture was stirred at room temperature for one hour and 30 minutes, then a mixture of 6.3 ml of dichloromethane and 12.12 ml of water was added. pH was adjusted to ph 6.3 with a saturated solution of sodium hydrogen carbonate, organic layer was concentrated to the rest of 6.4 g. Then 6.3 ml of acetone was added and evaporated to dryness. 1 ml of ethanol was added to precipitate crystals. To the resulting mixture 22 ml of n-hexane were added and stirring was continued at room temperature for one hour. The separated crystals were filtered and washed with 10 ml of a mixture of ethanol: n-hexane=1:9. Product was dried at 60° C. to obtain raw product (2.25 g), which was crystallized from 22.5 ml of i-propanol to obtain pure candesartan cilexetil in Form 1. Yield: 1.87 g

### Experiment 5 (Irbesartan)

23.25 g of trityl irbesartan was dissolved in 90 ml of dichloromethane. To a clear solution 90 ml of methanol, 1 ml of water and 0.15 ml of MSK was added. Reaction mixture was stirred at room temperature for 4 days, then 63 ml of dichloromethane and 121.2 ml of water was added. pH of reaction mixture was adjusted to ph 12 with NaOH 30%, layers were separated and water layer was washed with 63 ml of dichloromethane. pH of water layer was adjusted to ph 2 with 1 N HCl, suspension was filtered and washed with 50 ml of water and 30 ml of ethyl acetate and vacuum dried at 50° C. Yield: 11.37 g

### Experiment 6 (Candesartan Cilexetil)

50 g of trityl candesartan cilexetil are dissolved in a mixture of 145 ml of dichloromethane and 125 ml of methanol. The solution is cooled to approximately 5° C. and a solution of 7.6 ml of methanesulfonic acid in 25 ml of methanol is added within 15 to 20 min. The mixture is stirred at approximately 3° C. for 60 min. The reaction mixture is then added to a mixture of 100 ml of dichloromethane, 190 ml of water and 88 ml of satrated NaHCO₃ solution The pH of the mixture is adjusted to a pH of 6.4 to 6.5 with approximately 15 ml of saturated NaHCO₃ solution and the mixture is stirred for approximately 15 min. Layers are separated and the aqueous layer is extracted with 100 ml of dichloromethane. The combined dichloromethane layers are separated and extracted with 100 ml of water. The solution is concentrated in vacuo to approximately 108 g. 100 ml of acetone are added and the mixture is again concentrated in vacuo to about 100 g. 15 ml of ethanol are added to the residue. Seeds of candesartan cilexetil are added and the suspension is stirred for approximately 3 hours at ambient temperature. 7.5 ml of ethanol are added, the suspension is stirred for 1 hour and is then stored at 4° C. overnight. The suspension is warmed to room temperature and 350 ml of heptane are slowly added within 40 min. The suspension is stirred for 1 hour at ambient temperature and then for additional 3 hours in an ice bath. The product is then isolated by filtration, washed with 125 ml of heptane and dried in vacuum over night at ambient temperature. Yield 31.56 g (94.6%).

### INDUSTRIAL SCALE EXPERIMENT

13.6 kg candesartan is dissolved 43.3 kg DMF at temperature bellow 25° C.; thereto add 4.1 kg Three ethylamine and 10.4 kg trityl chloride and heat up to 60-65° C. After the reaction has completed the reaction mixture is poured into ethanol preheated to 50±2° C. and thereto water is added. Upon cooling pH is adjusted with aqueous HCl to 4.6. Isolated tritylcandesartan is dissolved in 50 kg DMF, and mixed at 25° C.; whereupon 2.2 kg potassium iodide, 4.4 kg potassium carbonate and 6.6 kg cilexetil chloride are added and mixture is heated to 60-65° C. until the reaction is completed. The product is isolated.

Reactor cooled bellow 5° C. is charged with 85.3 kg MeOH and 0.8 L water and 3.2 kg conc sulfuric acid, or equivalent of HCl, whereto above product is added. The suspension is mixed until the completion of the reaction. Thereafter the temperature is kept bellow 10° C. and 8.5 kg Three ethylamine and 21.6 L water are added. Product is washed with heptane and to the methanolic phase water is added, heated to 40-45° C. and upon cooling candesartan cilexetil is crystallized. A small amount of sulfuric acid may be added during the crystallization.

1. A process for the synthesis of a compound of formula (I), where the R represents an optionally substituted imidazole, dihidroimidazole or benzimidazole or amine from a compound of formula (II) where Y is a protecting group. 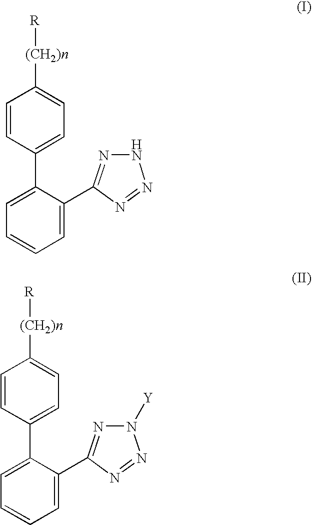
characterized in that the compound of formula (II) is reacted with a catalytic amount of an acid.

2. A process according to claim 1 wherein the acid is an organic acid.

3. A process according to claim 1 for the synthesis of candesartan cilexetil characterized by comprising following steps:
a) preparing a solution of trityl candesartan cilexetil in an alcohol, or alcohol water mixture;
b) mixing said solution with an acid, until the substantially all trityl candesartan cilexetil is converted to candesartan cilexetil;
c) adding an amine or ammonia to said solution of candesartan cilexetil to afford an ammonium or amine salt;
d) (optionally) adding a water immiscible solvent;
e) (optionally) separating layers; and
d) isolating the candesartan cilexetil by addition of an acid.

4. A process acceding to claim 3, wherein the amine is ammonia or trialkylamine.

5. A process according to claim 4, wherein the amine is Et₃N.

6. A process according to any of the claim 3, wherein the solution prepared in step a) is an aqueous solution.

7. A process according to any of the claim 3, wherein the acid used in step b) is a mineral acid.

8. (canceled)

9. A process according to claim 3, wherein the acid is sulfuric acid or hydrochloric acid.

10. A process for the synthesis of a compound of formula (I), where the R is such that the compound of formula (I) is selected form a group consisting of losartan, irbesartan and olmesartan medoxomil or salts thereof starting from a compound of formula (IIb). 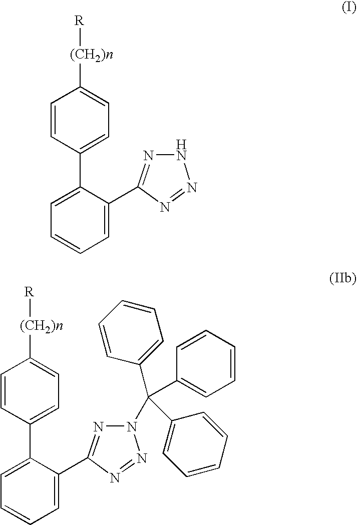
characterized in that the compound of formula (IIb) is dissolved in a solvent selected from chlorinated solvents, ethers, or alcohols, or mixture of them, optionally water is added and a catalytic quantity of a organic acids is used.

11. The process according to claim 1, the catalytic amount of from 1% to 75% molar ratio of organic acid to the starting compound is used.

12. A process according to claim 1, where acid is used in molar amount from 0.01 to 0.5 relative to the compound being deprotected

13. The process according to claim 1, where alcohol is methanol or ethanol.

14. (canceled)

15. (canceled)

16. The process according to claim 1, where the organic acid is selected from the group consisting of methane sulphonic acid, p-toluen sulphonic acid, pivalic acid, camphorsulphonic acid, trifluoracetic acid, ethanesulfonic acid, and benzensulfonic acid.

17. A pharmaceutical composition comprising a compound produced according to claim 1, and a pharmaceutically acceptable carrier.

18. (canceled)

19. A pharmaceutical composition comprising candesartan cilexetil produced according to claim 17.

20. A pharmaceutical composition according to claim 19, comprising another active ingredient.

21. A pharmaceutical composition according to claim 20, where another active ingredient is a diueretic.

22. A pharmaceutical composition according to claim 21, where a diueretic is hydrochlorotiazide.

23. (canceled)
